(19) United States
(12) Patent Application Publication
(10) Pub. No.: US 2024/0024071 A1
(43) Pub. Date: Jan. 25, 2024

(54) PREVENTING INJURY TO INFERIOR ALVEOLAR NERVE CAUSED BY DENTAL IMPLANT DRILLS DURING A DENTAL IMPLANT SURGERY

(71) Applicant: SEYEDSALAM RAHIMI, MAHABAD (IR)

(72) Inventor: SEYEDSALAM RAHIMI, MAHABAD (IR)

(21) Appl. No.: 18/103,162

(22) PCT Filed: Mar. 9, 2022
(86) PCT No.: PCT/IB2022/052076

§ 371 Date: Jan. 30, 2023

(51) Int. Cl. A61C 1/08; A61B 90/00

(52) U.S. Cl. CPC A61C 1/084; A61B 90/03; A61B 2090/035

(57) ABSTRACT

A system for preventing injury to inferior alveolar nerve caused by dental implant drills during a dental implant surgery. The system includes a dental implant drill, a clamp, an ohmmeter, a first wire, a second wire, and a processor. The dental implant drill is configured to make a hole in a mandible of a patient. The clamp is configured to be attached to a lower lip of the patient. The ohmmeter is connected to the dental implant drill and the clamp. The processor is configured to receive the measured electrical resistance between the dental implant drill and the clamp from the ohmmeter and turn off the dental implant drill by sending commands associated with turning off the dental implant drill to the dental implant drill when the measured electrical resistance between the dental implant drill and the clamp is less than a predetermined threshold.

The present disclosure generally relates to oral and maxillofacial surgery and, particularly, to dental implant surgeries. The present disclosure, more particularly, relates to a system for preventing injury to inferior alveolar nerve caused by dental implant drills during a dental implant surgery.

### BACKGROUND ART

The number of practitioners which perform implant surgery around the world has increased significantly over the last decades. As confidence is gained, practitioners or surgeons may tend to accept increasingly challenging cases and, reasonably, it should be expected that the incidence of problems and complications will increase. However, it remains a serious complication and many had reported the incidence, varies from 0 to 40%, of implant related inferior alveolar nerve (IAN) injuries. The damage may result from the traumatic local anesthetic injections or during the dental implant site osteotomy or placement. This damage may be considered as one of the worst and most unpleasant sufferings, from mild paresthesia to complete anesthesia and/or pain, for both the patient and the dentist. As a result, many functions such as speech, eating, kissing, make-up application, shaving, and drinking may negatively be affected.

The inferior alveolar nerve supplies the mandibular molar and premolar teeth and adjacent parts of the gingival. Its largest terminal branch emerges from the mental foramen as the mental nerve. Three nerve branches come out of the mental foramen. One innervates the skin of the mental area, and the other two proceed to the skin of the lower lip, mucous membranes, and the gingiva as far posteriorly as the second premolar. The incisive branch, a continuation of the inferior alveolar nerve mandibular, supplies the canine and incisor teeth. The inferior alveolar nerve may be considered as one of the most commonly injured nerves, followed by the lingual nerve. One of the main differences between inferior alveolar nerve injuries and other peripheral sensory nerve injuries is that the inferior alveolar nerve injury is predominantly iatrogenic and may not be recovered within the first eight weeks after injury. The closed injury may also occur that may delay diagnosis and treatment. The inferior alveolar nerve injury may significantly affect patient's quality of life and may also impose a great amount of stress to the dentist. Moreover, the iatrogenesis of inferior alveolar nerve injuries may compound the negative psychological effects of such injuries and when such an injury happens, the dentist should provide the appropriate care and treatment to the patient, as soon as possible.

One of the mechanical traumatic factors which may be a cause of inferior alveolar nerve is the intrusion of the dental implant drill into the mandibular canal which contains the inferior alveolar nerve. Such an intrusion of the dental implant drill may evoke direct mechanical injury i.e. pressure, encroach, transection, or laceration of the inferior alveolar nerve.

The most severe types of inferior alveolar nerve injuries are caused by dental implant drills. Sensory inferior alveolar nerve injuries made by dental implant drills may be caused by direct intraoperative (mechanical and chemical) and indirect postoperative trauma (ischemia and thermal stimuli). Many implants may be slightly longer than their corresponding implants (for drilling efficiency). Dental implant drill length may vary and may be understood by the surgeon because the specified length may not reflect an additional millimeter so called “y” direction. Damage to the inferior alveolar nerve may occur when the dental implant drill encroaches, transects, or lacerates the nerve.

Even after the accurate measurement of available bone, the nerve injury may occur as the result of over-penetration of the dental implant drill (direct intraoperative mechanical trauma) owing to low resistance of the spongy bone which may lead to slippage of the dental implant drill even when the implant surgery is done by experienced surgeons. There is, therefore, a need for a system that is able to prevent inferior alveolar nerve injuries caused by dental implant drills.

### SUMMARY OF THE DISCLOSURE

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a system for preventing injury to inferior alveolar nerve caused by dental implant drills during a dental implant surgery. In an exemplary embodiment, the system may include a dental implant drill, a clamp, an ohmmeter, a first wire, a second wire, and a processor.

In an exemplary embodiment, the dental implant drill may be configured to make a hole in a mandible of a patient. In an exemplary embodiment, the clamp may be configured to be attached to a lower lip of the patient. In an exemplary embodiment, the clamp may be made up of a conductive material. In an exemplary embodiment, the clamp may be made up of copper, iron, gold, aluminum, silver, or a combination thereof.

In an exemplary embodiment, the ohmmeter may be connected to the dental implant drill and the clamp. In an exemplary embodiment, the ohmmeter may be configured to measure an electrical resistance between the dental implant drill and the clamp. In an exemplary embodiment, the ohmmeter may include a display screen. In an exemplary embodiment, the display screen may be configured to show the measured electrical resistance between the dental implant drill and the clamp.

In an exemplary embodiment, the first wire may be interconnected between the ohmmeter and the dental implant drill. In an exemplary embodiment, the second wire may be interconnected between the ohmmeter and the clamp.

In an exemplary embodiment, the processor may be configured to receive the measured electrical resistance between the dental implant drill and the clamp from the ohmmeter. In an exemplary embodiment, the processor may further be configured to turn off the dental implant drill by sending commands associated with turning off the dental implant drill to the dental implant drill when the measured electrical resistance between the dental implant drill and the clamp is less than 400 ohms.

In an exemplary embodiment, the system may further include an alarm system. In an exemplary embodiment, the alarm system may be connected to the processor. In an exemplary embodiment, the alarm system may be configured to make an alert sound when the measured electrical resistance between the dental implant drill and the clamp is less than the predetermined threshold.

In an exemplary embodiment, the processor may further be configured to send commands associated with making the alert sound to the alarm system when the measured electrical resistance between the dental implant drill and the clamp is less than the predetermined threshold.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a view of a system for preventing injury to inferior alveolar nerve caused by dental implant drills during dental implant surgery, consistent with one or more exemplary embodiments of the present disclosure.

FIG. 1B illustrates another view of a system for preventing injury to inferior alveolar nerve caused by dental implant drills during dental implant surgery, consistent with one or more exemplary embodiments of the present disclosure.

FIG. 2A illustrates a view of a system for preventing injury to inferior alveolar nerve caused by dental implant drills during dental implant surgery in a scenario in which a dental implant drill encroaches and touches a mandibular canal of the patient, consistent with one or more exemplary embodiments of the present disclosure.

FIG. 2B illustrates another view of a system for preventing injury to inferior alveolar nerve caused by dental implant drills during dental implant surgery in a scenario in which a dental implant drill encroaches and touches a mandibular canal of the patient, consistent with one or more exemplary embodiments of the present disclosure.

FIG. 3 illustrates an example computer system in which an embodiment of the present disclosure, or portions thereof, may be implemented as computer-readable code, consistent with exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

The present disclosure is directed to exemplary embodiments of a system for preventing injury to inferior alveolar nerve caused by dental implant drills during a dental implant surgery. In an exemplary embodiment, the disclosed system may include a dental implant drill, a clamp, an ohmmeter, and a processor.

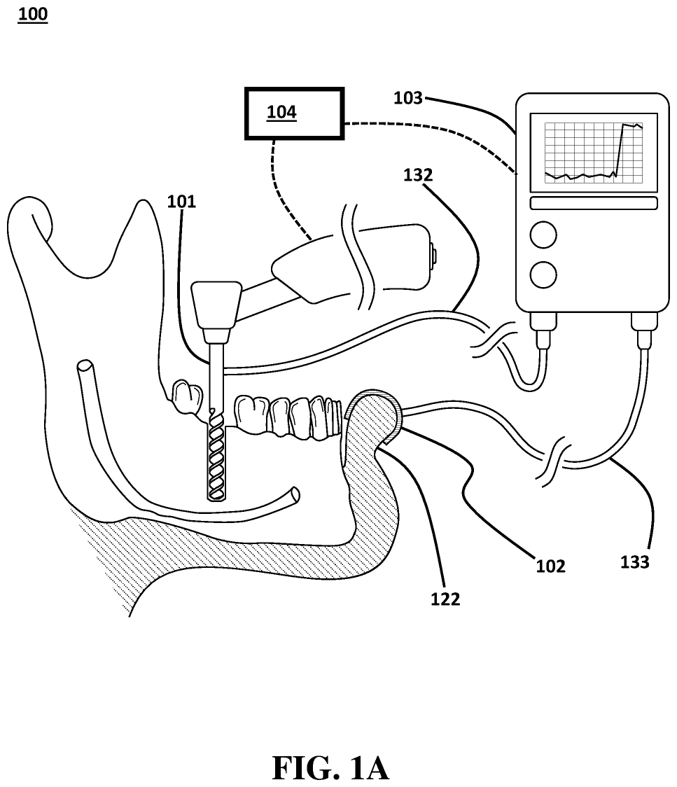

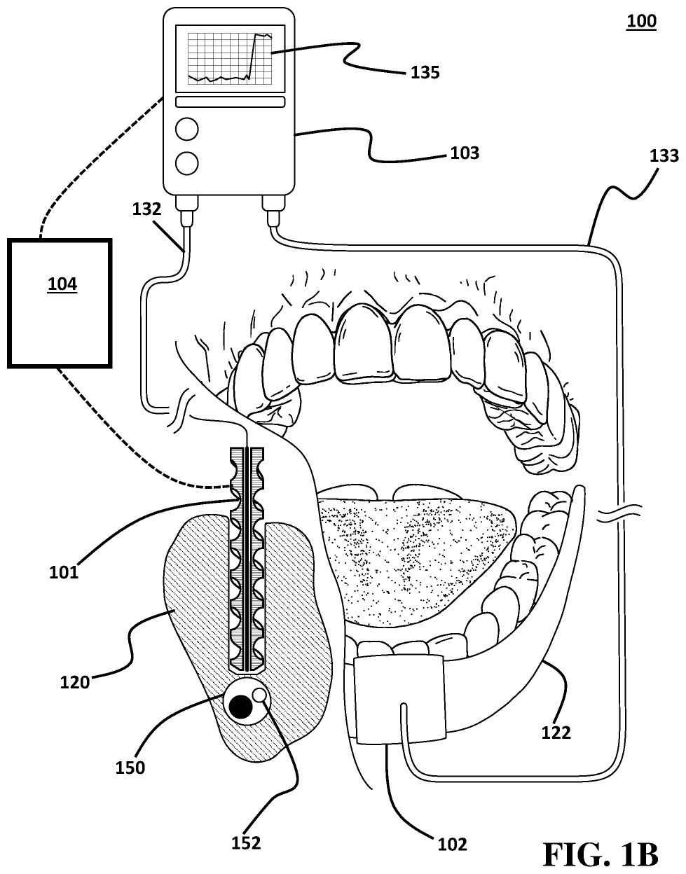

FIG. 1A shows a view of a system 100 for preventing injury to inferior alveolar nerve caused by dental implant drills during dental implant surgery, consistent with one or more exemplary embodiments of the present disclosure. FIG. 1B shows another view of system 100 for preventing injury to inferior alveolar nerve caused by dental implant drills during dental implant surgery, consistent with one or more exemplary embodiments of the present disclosure. As shown in FIG. 1A and FIG. 1B, in an exemplary embodiment, system 100 may include a dental implant drill 101. In an exemplary embodiment, dental implant drill 101 may be configured to make a hole in a mandible 120 of a patient. In an exemplary embodiment, it may be understood that this hole created by dental implant drill 101 may be later used to receive an implant. In an exemplary embodiment, the surgeon may later insert the implant into this hole. In an exemplary embodiment, a mandible of a patient may refer to a lower jaw of the patient. In an exemplary embodiment, system 100 may further include a clamp 102. In an exemplary embodiment, clamp 102 may be configured to be attached to a lower lip 122 of the patient. In an exemplary embodiment, clamp 102 may be made up of a conductive material. In an exemplary embodiment, a conductive material may refer to a material that is able to conduct electricity to a greater or lesser extent. These types of materials may allow electrons to flow freely and fluidly from one point to another if they are connected to a power source. For example, clamp 102 may be made up of copper, iron, gold, aluminum, silver, or a combination thereof. However, in different embodiments, clamp 102 may be made up of other conductive materials. In an exemplary embodiment, system 100 may further include an ohmmeter 103. In an exemplary embodiment, ohmmeter 103 may be an electrical instrument that is able to measure electrical resistance. In an exemplary embodiment, ohmmeter 103 may be connected to dental implant drill 101 and clamp 102. In an exemplary embodiment, ohmmeter 103 may be configured to measure an electrical resistance between dental implant drill 101 and clamp 102. In an exemplary embodiment, ohmmeter 103 may include a display screen 135. In an exemplary embodiment, display screen 135 may be configured to show the measured electrical resistance by the ohmmeter 135.

In an exemplary embodiment, system 100 may further include a first wire 132 and a second wire 133. In an exemplary embodiment, first wire 132 may be interconnected between ohmmeter 103 and dental implant drill 101. In an exemplary embodiment, second wire 133 may be interconnected between ohmmeter 103 and clamp 102. In an exemplary embodiment, system 100 may further include a processor 104. In an exemplary embodiment, processor 104 may be configured to receive the measured electrical resistance between clamp 102 and dental implant drill 101 from ohmmeter 103. In an exemplary embodiment, processor 104 may further configured turn off dental implant drill 101 by sending commands to dental implant drill 101 associated with turning off dental implant drill 101 when the measured electrical resistance between clamp 102 and dental implant drill 101 is less than a predetermined threshold. In an exemplary embodiment, the predetermined threshold may be in a range between 300 ohms and 500 ohms. In an exemplary embodiment, the predetermined threshold may be 400 ohms.

In an exemplary embodiment, it may be understood that when dental implant drill 101 is making a hole in mandible 120 (as shown in FIG. 1A and FIG. 1B), the electrical resistance between clamp 102 and dental implant drill 101 may be high, for example, when dental implant drill 101 is making a hole in mandible 120 (as shown in FIG. 1A and FIG. 1B), the electrical resistance between clamp 102 and dental implant drill 101 may be greater than 1000 ohms. Hence, when it is shown on display screen 135 of ohmmeter 103 that the electrical resistance between clamp 102 and dental implant drill 101 is higher than 1000 ohms, the surgeon may make sure that dental implant drill 101 is not in contact with a mandibular canal 150 of the patient. In an exemplary embodiment, it may be understood that once dental implant drill 101 touches mandibular canal 150 of the patient, the electrical resistance between clamp 102 and dental implant drill 101 may drop significantly due to the low electrical resistance of mandibular canal 150. For example, as soon as dental implant drill 101 touches mandibular canal 150, electrical resistance between clamp 102 and dental implant drill 101 may drop below 400 ohms. Then, when it is shown on display screen 135 of ohmmeter 103 that the electrical resistance between clamp 102 and dental implant drill 101 is less than 400 ohms, the surgeon may become aware that a head of dental implant drill 101 has touched mandibular canal 150 and, consequently, he or she may immediately turn off dental implant drill 101 or alternatively may withdraw dental implant drill 101 from the hole to prevent injury to inferior alveolar nerve 152 and also minimize the injury to mandibular canal 150 mandibular canal 150.

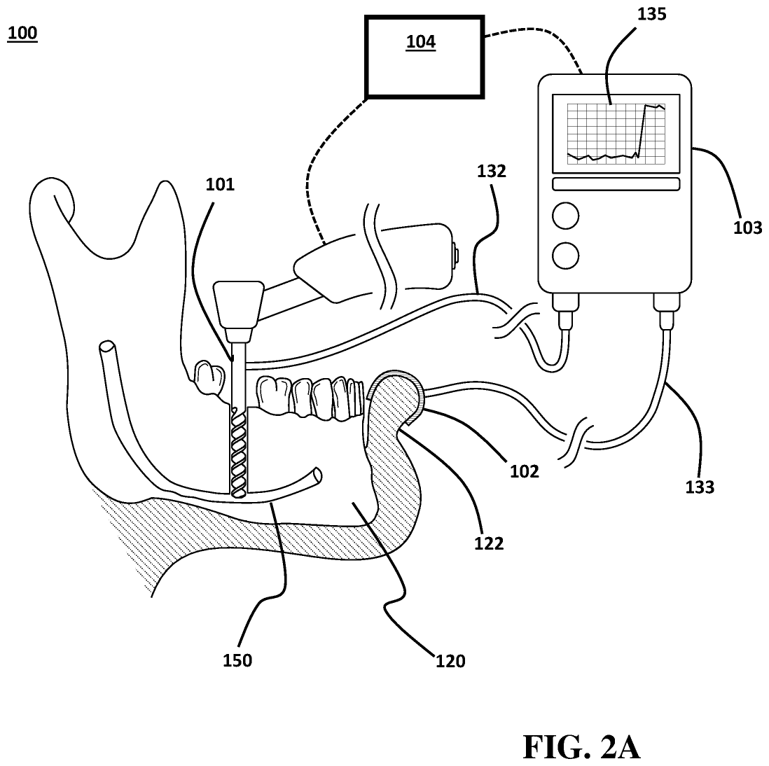

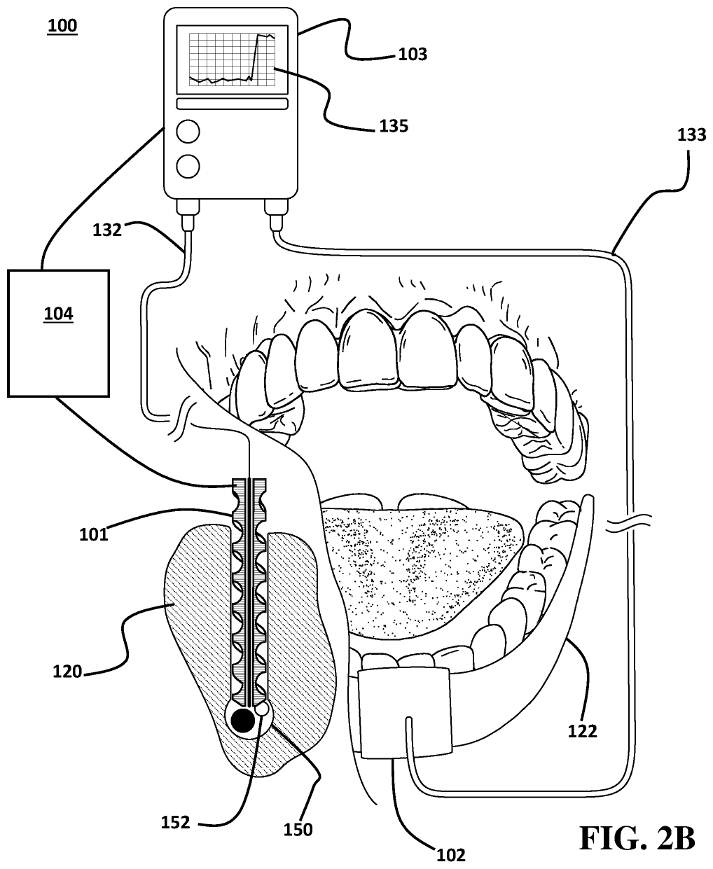

FIG. 2A shows a view of system 100 for preventing injury to inferior alveolar nerve caused by dental implant drills during dental implant surgery in a scenario in which dental implant drill 101 encroaches and touches mandibular canal 150 of the patient, consistent with one or more exemplary embodiments of the present disclosure. FIG. 2B shows another view of system 100 for preventing injury to inferior alveolar nerve caused by dental implant drills during dental implant surgery in a scenario in which dental implant drill 101 encroaches and touches mandibular canal 150 of the patient, consistent with one or more exemplary embodiments of the present disclosure.

In an exemplary embodiment, ohmmeter 103 may constantly measure the electrical resistance between clamp 102 and dental implant drill 101 during the period in which dental implant drill 101 is inside mandible 120 and send the measured electrical resistance to processor 104. In an exemplary embodiment, once dental implant drill 101 encroaches and touches mandibular canal 150 of the patient, the electrical resistance between dental implant drill 101 and clamp 102 may decrease significantly. In an exemplary embodiment, in this situation, the electrical resistance between dental implant drill 101 and clamp 102 may be less than a predetermined threshold. For example, when dental implant drill 101 is in direct contact with mandibular canal 150 of the patient, the electrical resistance between dental implant drill 101 and clamp 102 may be less than 400 ohms. Then, once the measured electrical resistance become less than the predetermined threshold, processor 104 may turn off dental implant drill 101 by sending commands associated with turning off dental implant drill 101 to dental implant drill 101.

In an exemplary embodiment, system 100 may further include an alarm system. In an exemplary embodiment, the alarm system may be connected to processor 104. In an exemplary embodiment, the alarm system may be configured to make an alert sound when the measured electrical resistance between the dental implant drill and the clamp is less than the predetermined threshold. In an exemplary embodiment, processor 104 may be configured to send commands associated with making the alert sound to the alarm system when the measured electrical resistance between the dental implant drill and the clamp is less than the predetermined threshold.

In an exemplary embodiment, system 100 may also be used for preventing injury to Schneiderian membrane during sinus lift surgery. For purpose of reference, it should be understood that Schneiderian membrane is the membranous lining of the maxillary sinus cavity. The Schneiderian membrane has osteogenic capability and participates in the formation of bone after the sinus floor has been lifted. However, its osteogenic role is weaker than that of the surrounding bony wall of the maxillary sinus. A sinus lift, aka sinus augmentation, is a surgery that makes it easier to place dental implants. Some people need dental implants but don't have enough bone support necessary for implants because of bone loss. A sinus lift can help as it increases the amount of bone in the upper jaw by adding bone to the space between your molars and premolars. To make room for the bone, the sinus membrane has to be moved upward or “lifted,” which gives the surgery its name. A dental specialist like an oral maxillofacial surgeon or a periodontist usually handle sinus lifts.

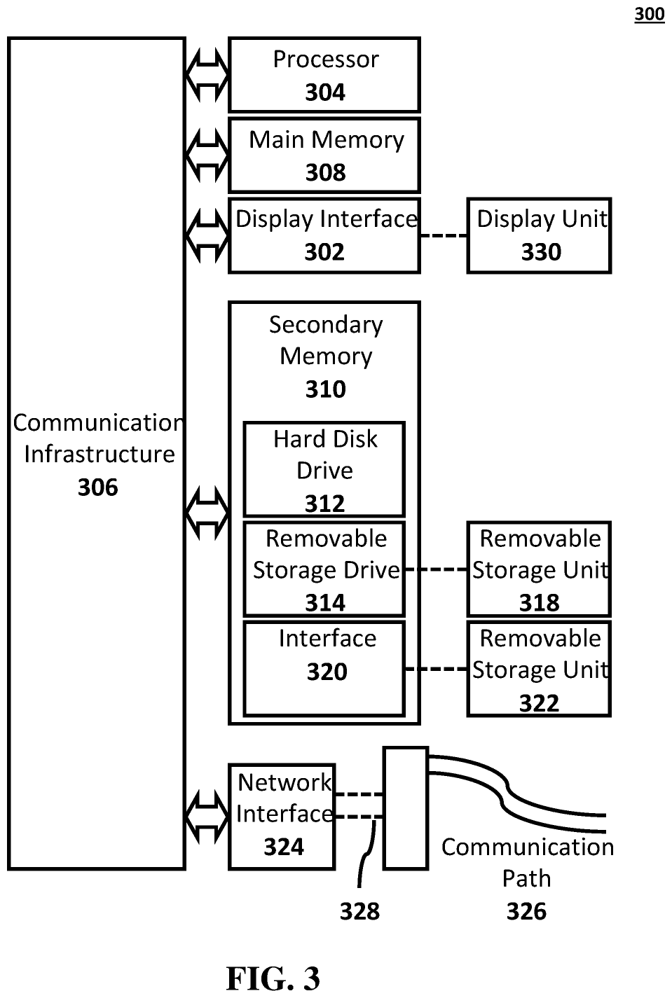

FIG. 3 shows an example computer system 300 in which an embodiment of the present disclosure, or portions thereof, may be implemented as computer-readable code, consistent with exemplary embodiments of the present disclosure. For example, processor 104 may be implemented in computer system 300 using hardware, software, firmware, tangible computer readable media having instructions stored thereon, or a combination thereof and may be implemented in one or more computer systems or other processing systems.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the disclosure is described in terms of this example computer system 300. After reading this description, it will become apparent to a person skilled in the relevant art how to implement the disclosure using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 304 may be a special purpose or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data.

In alternative implementations, secondary memory 310 may include other similar means for allowing computer programs or other instructions to be loaded into computer system 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320 which allow software and data to be transferred from removable storage unit 322 to computer system 300.

Computer system 300 may also include a communications interface 324. Communications interface 324 allows software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) are stored in main memory 308 and/or secondary memory 310. Computer programs may also be received via communications interface 324. Such computer programs, when executed, enable computer system 300 to implement different embodiments of the present disclosure as discussed herein. In particular, the computer programs, when executed, enable processor device 304 to implement the processes of the present disclosure. Accordingly, such computer programs represent controllers of computer system 300. Where an exemplary embodiment of the present disclosure may be implemented using software, the software may be stored in a computer program product and loaded into computer system 300 using removable storage drive 314, interface 320, and hard disk drive 312, or communications interface 324.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Ends 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective spaces of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A system for preventing injury to an inferior alveolar nerve of a patient caused by dental implant drills during a dental implant surgery, the system comprising:
a dental implant drill, the dental implant drill configured to make a hole in a mandible of a patient;
a clamp configured to be attached to a lower lip of the patient, the clamp made up of a conductive material, the clamp made up of copper, iron, gold, aluminum, silver, or a combination thereof;
an ohmmeter connected to the dental implant drill and the clamp, the ohmmeter configured to measure an electrical resistance between the dental implant drill and the clamp, the ohmmeter comprising a display screen, the display screen configured to show the measured electrical resistance between the dental implant drill and the clamp;
a first wire interconnected between the ohmmeter and the dental implant drill;
a second wire interconnected between the ohmmeter and the clamp; and
a processor, the processor configured to:
receive the measured electrical resistance between the dental implant drill and the clamp from the ohmmeter; and
turn off the dental implant drill by sending commands associated with turning off the dental implant drill to the dental implant drill responsive to the measured electrical resistance between the dental implant drill and the clamp being less than 400 ohms.

2. A system for preventing injury to an inferior alveolar nerve of a patient caused by dental implant drills during a dental implant surgery, the system comprising:
a dental implant drill, the dental implant drill configured to make a hole in a mandible of a patient;
a clamp configured to be attached to a lower lip of the patient, the clamp made up of a conductive material;
an ohmmeter connected to the dental implant drill and the clamp, the ohmmeter configured to measure an electrical resistance between the dental implant drill and the clamp;
a first wire interconnected between the ohmmeter and the dental implant drill;
a second wire interconnected between the ohmmeter and the clamp; and
a processor, the processor configured to:
receive the measured electrical resistance between the dental implant drill and the clamp from the ohmmeter; and
turn off the dental implant drill by sending commands associated with turning off the dental implant drill to the dental implant drill responsive to the measured electrical resistance between the dental implant drill and the clamp being less than a predetermined threshold.

3. The system of claim 2, wherein the predetermined threshold is 400 ohms.

4. The system of claim 3, wherein the clamp is made up of copper, iron, gold, aluminum, silver, or a combination thereof.

5. The system of claim 4, wherein the ohmmeter comprises a display screen, the display screen configured to show the measured electrical resistance between the dental implant drill and the clamp.

6. The system of claim 5, further comprising an alarm system connected to the processor, the alarm system configured to make an alert sound responsive to the measured electrical resistance between the dental implant drill and the clamp being less than the predetermined threshold, the processor configured to send commands associated with making the alert sound to the alarm system responsive to the measured electrical resistance between the dental implant drill and the clamp being less than the predetermined threshold.
